(19) United States
(12) Patent Application Publication
(10) Pub. No.: US 2013/0123643 A1
(43) Pub. Date: May 16, 2013

(54) MEASURING ARRANGEMENT FOR RECORDING A SPECTRUM, IN PARTICULAR FROM VITAL TISSUE

(71) Applicants: Holger Jungmann, Gelsenkirchen (DE); Michael Schietzel, Herdecke (DE)

(72) Inventors: Holger Jungmann, Gelsenkirchen (DE); Michael Schietzel, Herdecke (DE)

(21) Appl. No.: 13/639,362

(22) PCT Filed: Apr. 11, 2011
(86) PCT No.: PCT/EP11/01791

§ 371 Date: Jan. 22, 2013

(30) Foreign Application Priority Data
Apr. 12, 2010 (DE) 102010014702.8

(51) Int. Cl. A61B 6/00

(52) U.S. Cl. CPC A61B 6/00
USPC 600/476

(57) ABSTRACT

The invention relates to a measuring arrangement having a mobile spectrometer device which comprises a measuring head structure, wherein the measuring head structure is designed in such a way that, to carry out the spectrometric measurement, it can be placed on a portion of tissue, and a socket element which forms an inner receiving space into which the measuring head structure can be inserted, wherein a calibrating medium, produced from an opaque material, is arranged in a bottom region bounding the inner receiving space, and the socket element and the measuring head structure are designed such that, after the measuring head structure has been introduced into the socket element, the calibrating medium is sealed off in a light-tight manner from the surroundings.

The invention concerns a measuring assembly for collecting measuring signals indicative for a spectrum, especially from living tissue, for example for determining the presence, concentration, or composition of body liquids as well as of maybe only temporarily vascular-bound substances.

Movable spectrometer are known by which an analysis of temporarily vascular-bound substances can be done by applying this spectrometer to a corresponding tissue area of a living being to be examined and by recording, by this mobile spectrometer, the spectrum of reflected light emerging from the tissue. By means of the spectrum recorded in this way various substances in the examined tissue area can be detected. These spectrometers can be structured as classic spectrometers, in case of which the incident light is split by optical means and the intensity of the split light is measured by associating it to the wavelength. For avoiding movable parts the spectrometer can be formed in such a way that the light split according to its wavelength is led onto a CCD array and is analyzed by it as for its intensity.

The object of the invention is to create solutions by which it is possible to perform spectrometric measurements with particularly high reliability.

This task is solved according to the invention by a measuring assembly with:
a mobile spectrometer device that includes a measuring head structure formed in such a way that for performing the spectrometric measurement it can be applied to a tissue area, and
a bush element that constitutes an inner seat in which the measuring head structure can be inserted,
in a bottom area limiting the inner seat being arranged a calibration medium made of an opaque material, and
the bush element and the measuring head structure being formed in such a way that after introducing the measuring head structure into the bush element the calibration medium is light-tight isolated from the environment.

It is thus advantageously possible directly before the use of the spectrometer device to perform a calibration measurement, by which is guaranteed to a high degree that the is device works reliably in the relevant spectral range.

Preferably the calibration medium is releasably fastened in the bush element. The calibration medium is preferably configured in such a way that its composition as well as scattering characteristics substantially correspond to the properties of the skin. The calibration medium can be designed as an insert plate and is preferably configured to have a thickness of around 6 mm. Several such calibration plates can be retained in a set. The calibration plates are preferably configured to form a volume emitter, the scattering behavior of which corresponds to the behavior of human skin. These insert plates can be equipped each with determined substances, so that for special examinations the spectrometer can be calibrated for this substance on the basis of a opaque sample.

The measuring head structure of the spectrometer is preferably configured in such a way that it is coupled over a first light guide, a second light guide with a light source device and over a third light guide with a spectrometer device, these light guides ending in a support surface provided by the measuring head structure, and wherein the outlet positions of the light guides are coordinated in such a way that the distances of the outlet positions of the first and second light guide distinguish from the outlet position of the third light guide.

Preferably the measuring head structure is configured in such a way that the light guides enter substantially perpendicularly from behind into the support surface.

The distance of the outlet position of the first light guide from the outlet position of the third light guide is preferably greater than the distance of the outlet position of the second light guide from the outlet position of the third light guide.

The distance of the outlet position of the first light guide from the outlet position of the third light guide preferably corresponds to the distance of the outlet position of the first light guide from the outlet position of the second light guide.

The measuring head structure can be formed in such a way that the outlet positions of the light guide represent the vertices of a triangle, in which an interior angle defined between the legs extending towards the outlet position of the third light guide is in the range from 79° to 94°, preferably 89°.

A design of the measuring head structure that is particularly advantageous for measurements on vital human tissue is made in such a way that the distance of the outlet position of the first light guide from the outlet position of the third light guide amounts to 3.6 mm.
The distance of the outlet position of the second light guide from the outlet position of the third light guide preferably amounts to 2.3 mm.

The light source device is, according to a particular aspect of the present invention, configured in such a way that it includes two separate LED light sources, that each are associated to one of the light guides. The light guides are preferably iron-free multifilaments.

Further particulars and characteristics of the invention result from the following description in connection with the drawing. The figures show:

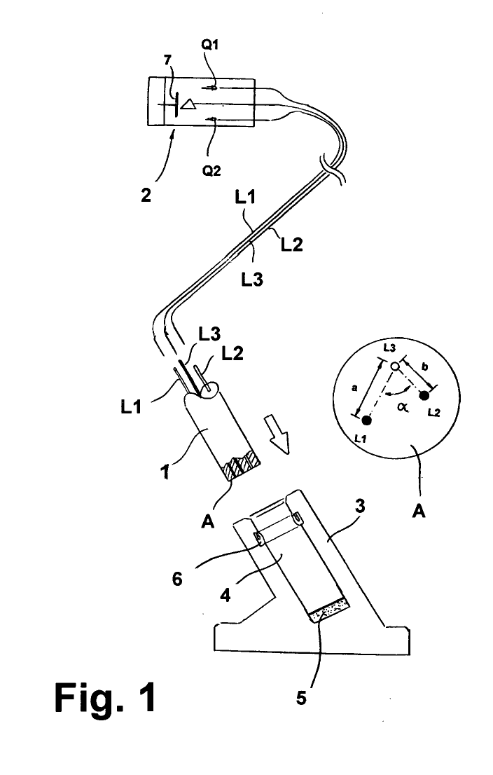

FIG. 1 a sketch to illustrate the structure of a mobile measuring device according to the invention.

FIG. 1 shows a measuring assembly for spectrometric analysis of vital tissue. The measuring assembly includes a measuring head 1. The measuring head 1 includes a first light guide L1 and a second light guide L2, which in the area of a basic device 2 are coupled to a light source device Q1, Q2. The measuring head 1 moreover has a third light guide L3 which is coupled with a spectrometer device 7 also provided in the basic device. These light guides L1, L2, L3 end in a support surface A provided by the measuring head structure 1, the outlet positions of the light guides L1, L2, L3 being coordinated in such a way that the distances a, b of the outlet positions of the first and second light guide L1, L2 distinguish from the outlet position of the third light guide L3 significantly, preferably by at least 0.4 mm.

The light guide L1s, L2 and L3 are integrated in such a way in the measuring head structure 1 that they substantially enter perpendicularly from behind into the support surface A. The support surface A or the end windows of the light guide L1, L2, L3 can be equipped with a seal or a thin window structure, so that the light guides are optically accessible and moreover are mechanically protected.

The distance of the outlet position of the first light guide L1 from the outlet position of the third light guide L3 is greater than the distance of the outlet position of the second light guide L2 from the outlet position of the third light guide L3. The distance of the outlet position of the first light guide L1 from the outlet position of the third light guide L3 corresponds roughly to the distance of the outlet position of the first light guide L1 from the outlet position of the second light guide L2.

The outlet positions of the light guide represent the vertices of a triangle, in which an interior angle defined between the legs extending towards the outlet position of the third light guide is in the range from 79° to 94°, preferably 89°.

The distance of the outlet position of the first light guide L1 from the outlet position of the third light guide L3 in the concrete embodiment preferably amounts to 3.6 mm. The distance of the outlet position of the second light guide L2 from the outlet position of the third light guide L3 preferably amounts to 2.3 mm.

The light source device includes two separate LED light sources Q1, Q2 which each are associated to one of the light guides L1, L2. The light guides L1, L2 are iron-free multifilaments and integrated into a cladding which is not shown here in detail with strain relief. The spectrometer device includes a CCD array 7 by which the spectral distribution of the intensity of the light detected by the third light guide L3 can be determined by associating it to the wavelength.

The support surface is formed preferably substantially circular or slightly elliptical. The outlet positions of the light guide L1, L2, L3 are preferably established in such a way that the centroid of a triangle defined accordingly by these outlet positions substantially coincides with the centroid of the support surface.

The measuring head structure 1 is formed in such a way that it can be applied to a tissue area for performing the spectrometric measurement. The measuring assembly includes a bush element 3 that constitutes an inner seat 4 in which the measuring head structure 1 can be inserted. In a bottom area limiting the inner seat 4 a calibration medium made of an opaque material 5 is arranged. The calibration medium 5 constitutes a volume emitter.

The bush element 3 and the measuring head structure 1 are formed in such a way that after introducing the measuring head structure 1 into the bush element 3 the calibration medium 5 is light-tightly isolated from the environment. For this purpose moreover a sealing device 6 is provided.

In the embodiment shown here the calibration medium 5 is releasably fastened in the bush element 3.

The calibration medium 5 is configured in such a way that it in its composition as well as its scattering characteristics corresponds substantially to the properties of the skin. The calibration medium 5 is designed as an insert plate and presents a thickness of around 6 mm.

For a test or calibration measurement the measuring head structure is inserted into the bush element 3 to such an extent that the support surface A rests on the calibration medium 5.

The calibration or gauging of the spectrometer device or of the attached signal processing device can largely be done automatically using a signal processing procedure preferably deposited in the measuring instrument by doing a measurement after the insertion of the measuring head 1 into the bush element 3 and by classifying, by a corresponding interface, this measurement as calibration, gauge, or reference measurement.

1. A measuring assembly with:
a mobile spectrometer device that includes a measuring head structure formed in such a way that for performing the spectrometric measurement it can be applied to a tissue area,
a bush element that constitutes an inner seat in which the measuring head structure can be inserted, and
a calibration medium in a bottom area limiting the inner seat, and the bush element and the measuring head structure (1) are formed in such a way that after introducing the measuring head structure into the bush element the calibration medium is light-tight isolated from the environment.

2. The measuring assembly according to claim 1, wherein the calibration medium is made of a opaque material.

3. The measuring assembly according to claim 1, wherein the calibration medium is releasably fastened in the bush element.

4. The measuring assembly after claim 1, wherein the calibration medium is configured in such a way that it in its composition as well as its scattering characteristics corresponds substantially to the properties of human skin tissue.

5. The measuring assembly after claim 1, wherein the calibration medium is designed as an insert plate and presents a thickness of around 6 mm.

6. The measuring assembly after at claim 1, wherein the measuring device includes a light source device, a spectrometer device, and a measuring head structure, wherein the measuring head structure is coupled with the light source device over a first light guide and a second light guide as well as with the spectrometer device over a third light guide, and in which these light guides end in a support surface provided by the measuring head structure, and wherein the outlet positions of the light guide are coordinated in such a way that the distances of the outlet positions of the first and second light guide distinguish from the outlet position of the third light guide.

7. The measuring assembly according to claim 6, wherein the light guides substantially enter perpendicularly from behind into the support surface.

8. The measuring assembly according to claim 6, wherein the distance of the outlet position of the first light guide from the outlet position of the third light guide is greater than the distance of the outlet position of the second light guide from the outlet position of the third light guide.

9. The measuring assembly according to claim 1, wherein the light guides and are integrated into a cladding, and that a connection arrangement is provided for the releasable optic connection of the light guides to the basic device.
